(19) United States
(12) Patent Application Publication
(10) Pub. No.: US 2022/0395210 A1
(43) Pub. Date: Dec. 15, 2022

(54) DIFFERENTIAL VOLTAGE MEASURING SYSTEM FOR MEASURING THE BREATHING ACTIVITY OF A PATIENT

(71) Applicant: Siemens Healthcare GmbH, Erlangen (DE)

(72) Inventor: Ulrich BATZER, Spardorf (DE)

(73) Assignee: Siemens Healthcare GmbH, Erlangen (DE)

(21) Appl. No.: 17/837,174

(22) Filed: Jun. 10, 2022

(30) Foreign Application Priority Data
Jun. 15, 2021 (EP) 21179522.4

(51) Int. Cl. A61B 5/277; A61B 5/00

(52) U.S. Cl. CPC A61B 5/277; A61B 5/725; A61B 5/7264; A61B 5/0024

(57) ABSTRACT

The differential voltage measuring system has a number of signal measuring circuits, each having a capacitive sensor element for capturing a measurement signal relating to the patient. The differential voltage measuring system further has a signal processing apparatus for determining at least one bioelectrical signal from the measurement signals and a computer unit which is configured to ascertain, on the basis of the at least one bioelectrical signal, and to provide, an item of breathing information, said breathing information indicating a breathing activity of the patient.

### CROSS-REFERENCE TO RELATED APPLICATION(S)

The present application claims priority under 35 U.S.C. § 119 to European Patent Application No. EP 21179522.4, filed Jun. 15, 2021, the entire contents of which are incorporated herein by reference.

### FIELD

Embodiments of the present invention relate to a differential voltage measuring system for measuring a breathing activity of a patient. Embodiments of the present invention also relate to a method for determining a breathing activity of a patient with a differential voltage measuring system.

### BACKGROUND

For imaging processes, it is necessary that the portion being recorded is at rest. For recordings in the region of the thorax, including cardiological recordings, apart from the movement of the heart, above all, the movement due to breathing is disruptive.

For the measurement of a heart activity, voltage measuring systems designed for measuring bioelectrical signals have become established. These are used as standard practice in the medical field for measuring electrocardiograms (ECG), electroencephalograms (EEG) or electromyograms (EMG). For this purpose, sensors which must be fastened to the body of the patient can be used. Alternatives to this are so-called capacitive measuring systems with which an ECG signal can be tapped off purely capacitively without directly contacting the patient with the sensor, in particular through the clothing of the patient. For the patient this is particularly comfortable, since the capacitive ECG measurement can take place without the placement or fastening of individual sensors. For this purpose, it is known to integrate the sensor equipment into the surface of a patient support of, for example, an imaging system or into an underlay mat or a (seat) backrest so that the voltage measurement can take place as soon as the patient is positioned on the patient support or the seat. An apparatus of this type is described, for example, in the German patent application DE 10 2015 218 298 B3. The information obtained in this way regarding the heart activity can be fed to an imaging system that is connected, in order to adapt the imaging to the heart activity and to eliminate or at least to take into account interference effects due to the heartbeat of the patient during the imaging.

### SUMMARY

In order to reduce interference effects in the imaging due to a breathing activity of the patient, the possibility always exists of having the patient hold his breath. However, this is not possible with all patients, it is often wrongly executed, and sometimes leads to severe changes in the heartbeat, making synchronization therewith more difficult.

It is therefore desirable to capture the breathing activity of the patient with measuring technology as during the measurement of heart activity and to take account thereof in the imaging. For this purpose, measuring systems from the prior art such as respiratory sensing belts or camera or radar-based respiratory detection systems are known. Such systems have the disadvantage, however, that additional components have to be provided and implemented. In addition, this has the consequence that during imaging, two signals from different measuring systems, the system for measuring a heart activity and the breathing detection system, must be taken into account and finally must be synchronized in some form, which makes the matching of the imaging to the measurement signals more difficult.

Embodiments of the present invention provide a remedy here and to provide a measuring system and associated methods which enable an improved measurement of a breathing activity of a patient and in particular are readily compatible with the measurement of a heart activity of the patient.

Embodiments of the present invention provide a differential voltage measuring system for measuring a breathing activity of a patient and/or a method for measuring a breathing activity of a patient. Preferred and/or alternative, advantageous embodiments are the subject matter of the following description. The inventive solution to the problem is described below, both in relation to the claimed systems and also in relation to the claimed method. Features, advantages or alternative embodiments/aspects mentioned herein are also transferable similarly to the other claimed subject matter and vice versa. In other words, the object-related claims (which are directed, for example, to a system) can also be further developed with the features described or claimed in conjunction with a method and vice versa. The corresponding functional features of the method can thereby be embodied by way of corresponding physical modules.

Furthermore, the inventive solution to the problem is also described in relation to methods for adapting trained functions. Herein, features and alternative embodiments/aspects of data structures and/or functions in methods and apparatuses for determining a breathing activity can be transferred to analogous data structures and/or functions in methods and apparatuses for adapting a trained function. Herein, analogous data structures can be characterized, in particular, by the use of the qualifier “training”. Furthermore, the trained functions used in methods and systems for determining a breathing activity of a patient can have been adapted and/or provided by methods and apparatuses for adapting trained functions.

According to one aspect, a differential voltage measuring system for determining a breathing activity of a patient is provided. The voltage measuring system has: a number of signal measuring circuits, each having a capacitive sensor element for capturing a measurement signal relating to the patient, a signal processing apparatus for determining at least one bioelectrical signal from the measurement signals; and a computer unit which is configured to ascertain on the basis of the at least one bioelectrical signal, and to provide, an item of information, said item of information indicating a breathing activity of the patient (also referred to hereinafter as breathing information).

Differential voltage measuring systems are known per se to the person skilled in the art according to their basic functioning, so that a detailed explanation is omitted here. In particular, they can conventionally be configured, in particular, to provide electrocardiograms (ECG), electroencephalograms (EEG) or electromyograms (EMG).

According to some embodiments, the capacitive sensor elements can each have a capacitive sensor electrode which is galvanically separated from the patient or is electrically connected to the patient, for example, by way of the clothing via a high impedance, preferably with an impedance of more than 1 MOhm. It is even advantageous to design the uppermost layer of the capacitive sensor element to be conductive and thus to provide a weak galvanic connection. An advantage of a weak galvanic connection lies therein that a better signal transference for signal components with low frequencies is achieved. If a capacitance is connected in parallel with an ohmic resistance, this arrangement forms a lower impedance for the low frequencies than the capacitance alone. In addition, such a connection enables a discharging in the context of an ESD protection. Furthermore, in the event that a maximum ohmic resistance of, for example, 100 MOhm is specified, the requirements placed on the input impedance of the signal measuring circuit connected to the sensor element or the input connection of said circuit are reduced since the ohmic resistance of the sensor electrode forms a voltage divider with the input impedance. Such a case exists if the clothing of the patient is correspondingly sufficiently electrically conductive and, in particular, does not consist of wool.

The capacitive sensor elements can each be connected via corresponding useful signal paths to the associated signal measuring circuit. The useful signal paths can be configured as sensor lines and/or individual cables. The signal measuring circuits can each be designed as an amplifier circuit and comprise, for example, an operational amplifier which can be designed in particular as followers. The signal measuring circuits can also each function as input buffers for the measurement signals captured with the sensor elements and can be designated as such. According to some exemplary embodiments, the signal measuring circuits can have an AD converter which is designed to digitize the measurement signals received by the sensor elements. Alternatively, an AD converter can also be constructed in the signal processing apparatus or can be provided separately. The measurement signals are, in particular, electrical signals which are modulated by a behavior or by properties of a human or animal patient and thus indicate and/or show these properties or behaviors. Indicate and/or show can therein mean, in particular, that it is possible to conclude these properties or behaviors with the signal and/or data processing from the measurement signals. In this case, a property and/or a behavior can be, in particular, a heart activity or a breathing activity.

The differential voltage measuring system can have, in particular, at least a first capacitive sensor element and a second capacitive sensor element for measuring the measurement signals. Furthermore, the differential voltage measuring system according to embodiments of the present invention preferably has at least one third capacitive sensor element with a third useful signal path for a potential equalization between a measurement object, for example a patient, and the differential voltage measuring system. The third, preferably provided useful signal path provides for a potential equalization between the patient and the capacitive differential voltage measuring system. Therein, the capacitive sensor of the third useful signal path is preferably applied to the right leg of the patient, from which the designation Right Leg Drive path originates. In principle, however, this third potential can also be captured at another site on the patient.

The measurement signals captured by way of the sensor elements and the associated signal measuring circuits are input into the signal processing apparatus which is configured to process the measurement signals further to one or more bioelectrical signals. In other words, a bioelectrical signal therefore represents a signal generated on the basis of the measurement signals. The bioelectrical signals generated in this way are configured such that they indicate a breathing activity of the patient and/or are modulated by the breathing activity of the patient. The signal processing apparatus can thus be considered to be a preprocessing stage which is connected upstream of the ascertainment of the item of breathing information in the computer unit. For the determination of such bioelectrical signals, the signal processing apparatus can be configured, for example, to select suitable measurement signals from corresponding sensor elements, to identify and extract and/or to amplify relevant measurement signal components (in particular relevant frequency components) and/or to amplify and/or to dampen out and/or eliminate irrelevant signal components, in particular interference components. For the determination of one or more bioelectrical signals, the signal processing apparatus can be configured accordingly using hardware and/or software components. For example, the signal processing apparatus can bring suitable filter and/or amplifier elements and/or computer program products into effect which implement these elements virtually.

The bioelectrical signal(s) is/are transferred into a computer unit which is configured to ascertain an item of breathing information regarding the patient from the bioelectrical signal(s). The computer unit can therein be configured as part of the signal processing apparatus. Conversely, the signal processing apparatus can be configured as part of the computer unit. The computer unit can bring into effect, in particular, a computer program product which is designed and/or adapted for ascertaining the item of breathing information. According to some exemplary embodiments, the item of breathing information can comprise an item of information relating to the inhalation and exhalation processes of the patient, particularly in semi-real time. According to further exemplary embodiments, the item of breathing information can additionally or alternatively comprise an item of information regarding a local breathing component of the patient which reveals which body part the breathing of the patient is currently affecting and in which way. The computer unit can have, for example, one or more processors. The processors can be configured as a central processing unit (CPU). Furthermore, the computer unit can comprise a storage facility for at least temporary storage of data, signals and/or computer program products.

The proposed differential voltage measuring system has as its basis the recognition that bioelectrical signals that are modulated by the breathing activity of the patient can be derived from a differential voltage measurement as is already used for determining heart activity. By way of the targeted acquisition of these signals, the same setup as used for determining the heart activity of the patient can, in principle, be used for determining the breathing activity. Thus a solution is provided which is simple in use, is cost-effective with regard to additional setup costs, and with which a breathing activity of a patient can be obtained, for example, for an imaging process adapted thereto. In particular, the proposed voltage measuring system offers the possibility of capturing information regarding heart and breathing activity in a concentrated manner and of providing it to a connected imaging process as integrated input information, for instance, in the form of a trigger signal.

According to some exemplary embodiments, the signal processing apparatus is designed for determining at least two different bioelectrical signals from the measurement signals. Accordingly, the computer unit is configured to take account of each of the at least two different bioelectrical signals during the ascertainment of the item of breathing information.

In other words, two different signals are thus utilized for determining the item of breathing information, which can ensure an improved and more reliable breathing detection. The different bioelectrical signals can each be realized by way of different signal processing paths in the signal processing apparatus. For example, for different bioelectrical signals, different measurement signals can be selected. Additionally or alternatively, for different bioelectrical signals, different signal components can be extracted from the measurement signals with corresponding filter modules.

According to some exemplary embodiments, the at least one bioelectrical signal comprises a beat-to-beat heart rate of the patient, an ECG vector of the patient and/or a muscle activity of the patient.

The use of the beat-to-beat heart rate as a bioelectrical signal is based upon the recognition that on inhalation, the heart beats faster than on exhalation. For determining such a bioelectrical signal, the signal processing apparatus can comprise, in particular, a filter module for filtering the measurement signals, said filter module being configured for filtering frequencies between 1 Hz and 40 Hz.

The use of an ECG vector as a bioelectrical signal for determining the item of breathing information is based upon the recognition that the heart rotates in the body during breathing. In the differential voltage measuring system, this is apparent in that the amplitude of the measurement signals is displaced between the sensor elements and/or the measurement signals change in a location-dependent manner. For the measurement of the ECG vector, therefore, measurement signals are combined from at least two, preferably three or more sensor elements that are arranged at different locations in relation to the patient. Essentially, similar information is extracted from the measurement signals for determining the ECG vector as for the beat-to-beat heart rate, but spatially resolved. Accordingly, for determining a corresponding bioelectrical signal, the signal processing apparatus can comprise, in particular, a filter module for filtering the measurement signals, said filter module being constructed for filtering frequencies between 1 Hz and 40 Hz.

The use of an ECG vector as a bioelectrical signal for determining the item of breathing information is based upon the recognition that through the measurement of the muscle activity, particularly in the region of the ribcage of the patient, conclusions can be drawn directly regarding the breathing activity of the patient. Accordingly, the differential voltage measuring system can have at least one sensor element that is suitable and, in particular, is arranged so that it contacts the ribcage of the patient in a region in which the muscle activity on breathing is measurable. The measurement signals indicating the muscle activity are characterized by elevated signal components at 10-100 Hz. Accordingly, for determining a corresponding bioelectrical signal, the signal processing apparatus can comprise, in particular, a filter module for filtering the measurement signals, said filter module being constructed for filtering frequencies between 10 Hz and 100 Hz.

According to some exemplary embodiments, the at least one bioelectrical signal can be configured such that it indicates a capacitive coupling between the sensor element and the patient. In order to provide a bioelectrical signal of this type, the signal processing apparatus can have a reference signal capture apparatus and a comparator module. The reference signal capture apparatus is configured to capture a reference signal. The comparator module is configured to ascertain, on the basis of the reference signal, a corrected measurement signal and, on the basis of the corrected measurement signal, to provide, as the bioelectrical signal, a signal from which a capacitive coupling between at least one sensor element and the patient can be derived.

The use of a bioelectrical signal indicating a capacitive coupling between the sensor element and the patient as the basis for ascertaining the item of breathing information is based upon the recognition that through the breathing of the patient, a loading by the body of the patient on an underlay becomes displaced in a location-dependent manner. As a consequence thereof, with the capacitive sensor elements used, a coupling between the patient and the sensor electrode which is measurable, in principle, by way of a modulation of the respective measurement signal, changes. However, this modulation is overlaid by other effects, such as a change in external electromagnetic fields. In order to eliminate such overlaying effects, it is provided to adjust the measurement signals with a reference signal acting, in particular from outside, upon the sensor elements and/or to filter them against such a reference signal in order thereby to obtain a correspondingly corrected measurement signal. The change in the electric fields on individual sensor elements can thus always be placed into relation with a reference value and thus merely reflects the change in the measurement signals of this sensor element by way of the changed pressure from the patient on the sensor element. The reference signals are caused by effects that are overlaid on the measurement object that is actually to be captured.

The reference signal capture apparatus can, for example, have additional sensors for measuring the reference signal and/or can be configured to capture the reference signal by measurement of electrical currents or voltages on or in the differential voltage measuring system itself. The reference signal capture apparatus can comprise, for example, the aforementioned third capacitive sensor element.

According to some exemplary embodiments, the comparator module comprises a reference formation filter which is designed to filter the reference signal, and an adaptive filter unit which is designed to filter the measurement signal on the basis of the filtered reference signal, and thus to ascertain the corrected measurement signal.

The reference formation filter can be configured, for example, as a low-pass filter. Advantageously, high-frequency portions of the reference signal which, in contrast to a measurement with electrodes, in a measurement with capacitive measuring sensors, as far as frequency and phase are concerned, are not well suited to the interference components in the measurement signal, are suppressed. After this filtering procedure, the reference signal comprises, above all, low frequency components which match well to the reference signal components on the measurement signal.

The input of the adaptive filter element is connected downstream of the reference formation filter, so that the comparator module can further process the reference signal already filtered by the reference formation filter as the reference signal. In this adaptive filter procedure, an estimation of a reference signal component of the measurement signal takes place on the basis of the reference signal filtered by the reference formation filter. This reference signal component is then removed from the measurement signal. The adaptation of the adaptive filter can take place by iterative or recursive optimization processes. In this way, in particular, low frequency portions of the reference signal, that is, the filtered reference signal, is used for an adaptive filter process to remove reference signal components and to isolate a modulation of the signal by way of a changed contact of the patient with the sensor element.

According to some exemplary embodiments, the signal processing apparatus has a prefilter, in particular in the form of a comb filter which is configured to extract frequencies of 50 Hz, 60 Hz and/or their harmonics from the measurement signal and/or the reference signal.

The use of a reference signal and the analysis of the measurement signal at these frequencies has the advantage that in an electrically powered environment, electric fields of the mains power supply are always present for this analysis at 50 Hz and/or 60 Hz and their harmonic frequencies. It is therefore not necessary to apply a separate reference signal.

According to some exemplary embodiments, the at least one bioelectrical signal is a spatially resolved signal. This can mean, in particular, that signal components can be traced back to individual sensor elements and thus enable a statement to be made regarding conditions at the location of the respective sensor. Spatially resolved bioelectrical signals can be, in particular, the ECG vector, the muscle activity signal and/or the signal indicating a capacitive coupling.

According to some exemplary embodiments, the differential voltage measuring system further has a number of pressure-sensitive measuring elements, each of which is configured for capturing a pressure signal indicating a local pressure loading by the patient on an underlay. Furthermore, the differential voltage measuring system has a secondary signal processing apparatus for determining at least one, in particular spatially resolved, pressure loading signal from the pressure signals, the computer unit being further configured to provide an item of information indicating a breathing activity on the basis of the pressure loading signal.

Through the provision of pressure-sensitive measuring elements and the evaluation of the measurement signals captured thereby, an information item orthogonal to the differential voltage measurement can be obtained with which the item of breathing information can be determined more exactly. Since the weight of the patient is displaced during the breathing, different measuring elements are compressed to different degrees. In particular, thereby a local breathing component of the patient can be determined that yields not only the breathing frequency, but also indicates which body part is affected by the breathing.

The underlay can be configured, for example, as a patient mat. The pressure-sensitive measuring elements and/or the capacitive measuring elements can therein be arranged on and/or in the underlay and/or incorporated into it. According to some exemplary embodiments, the underlay is included in the differential voltage measuring system.

According to some exemplary embodiments, at least one of the capacitive sensor elements has a mechanical mounting which is configured to support a sensor electrode of the corresponding sensor element, in particular in the underlay, one of the pressure-sensitive measuring elements additionally being accommodated in the mechanical mounting.

By way of this embodiment, pressure-sensitive measuring elements can easily be integrated and additionally provide a complementary pressure signal from the location of the capacitive sensor element, which can allow, for example, a verification of the measurement signals by way of an equalization with a corresponding pressure signal. Additionally or alternatively, other pressure-sensitive measuring elements can however be provided, independently of the capacitive sensor elements.

According to some exemplary embodiments, the mechanical mounting has a support element which can be deformed, in particular elastically and is preferably made of foam material and supports the sensor electrode. The pressure-sensitive measuring element comprises two conductive layers, one layer being arranged on a first side of the support element and the other layer being arranged on a second side of the support element opposite the first side. The differential voltage measuring system further comprises a pressure measuring circuit which is designed to apply a constant current to the conductive layers and to capture the voltage drop across it as a pressure signal. Also possible, however, is a separate pressure-sensitive measuring element under or on the support element.

According to some exemplary embodiments, the differential voltage measuring system also has an output unit which is designed to output an item of information indicating a breathing activity and at least one of the bioelectrical signals, the output bioelectrical signal indicating a heartbeat of the patient. Accordingly, consolidated information regarding both the heart activity and also the breathing activity of the patient, which can be used, for example, in order to synchronize downstream systems and/or applications with the heart activity and/or breathing activity, is provided by the differential voltage measuring system.

According to some exemplary embodiments, the computer unit comprises a Kalman filter which is configured, on the basis of the signals input into the computer unit (that is, in particular, the bioelectrical signal(s) and/or the pressure loading signal) to ascertain and provide the item of information indicating the breathing activity of the patient. The use of a Kalman filter therein enables, in particular, the item of breathing information to be estimated in real time from the input signals.

According to some exemplary embodiments, the computer unit is configured to apply a trained function to the signals input into the computer unit, said trained function being configured, on the basis of the signals input into the computer unit, to ascertain and provide an item of information indicating the breathing activity of the patient.

In general, a trained function maps input data to output data. For this purpose, the output data can depend, in particular, upon one or more parameters of the trained function. The one or more parameters of the trained function can be determined and/or adapted by a training process. The determination and/or the adaptation of the one or more parameters of the trained function can be based, in particular, upon a pair made from training input data and associated training output data, the trained function being applied to the training input data to generate intermediate output data. In particular, the determination and/or the adaptation can be based upon a comparison of the intermediate output data and the training output data. In general, a trainable function, i.e. a function with as yet non-adapted parameters, is also designated a trained function. Other expressions for trained function are trained mapping rule, mapping rule with trained parameters, function with trained parameters, algorithm based upon artificial intelligence, algorithm of machine learning.

According to some exemplary embodiments, the trained function comprises an artificial neural network. In place of the expression “neural network”, the expression “neural net” can also be used. A neural network is fundamentally constructed like a biological neural network—for instance a human brain. In particular, an artificial neural network comprises an input layer and an output layer. It can also comprise a plurality of layers between the input layer and the output layer. Each layer comprises at least one, preferably a plurality of, nodes. Each node can be understood as a biological processing unit, e.g. as a neuron. In other words, each neuron corresponds to an operation that is applied to input data. Nodes of a layer can be connected by way of edges or connections to nodes of other layers, in particular by way of directed edges or connections. These edges or connections define the data flow between the nodes of the network. The edges or connections are associated with a parameter, commonly designated the “weight” or “edge weight”. This parameter can regulate the importance of the output of a first node for the input of a second node, the first node and the second node being connected by way of an edge. In particular, a trained function can also have a deep neural network (or deep artificial neural network). In particular, a neural network can be trained. In particular, the training of a neural network is carried out on the basis of the training input data and the associated training output data according to a supervised learning technique, wherein the known training input data is input into the neural network and the intermediate output data generated by the network is compared with the associated training output data. The artificial neural network learns and adapts the edge weights for the individual nodes independently for as long as the intermediate output data of the last network layer does not sufficiently correspond to the training output data.

According to one aspect, a computer-implemented method for providing a trained function is provided. The method has a plurality of steps. A first step is directed to a provision of one or more bioelectrical training signals and, optionally, a training pressure loading signal of a patient as training input data. A further step is directed to a provision of an item of training breathing information which indicates a breathing activity of the patient corresponding to the bioelectrical training signals and/or training pressure loading signals. A further step is directed to a use of the trained function on the training input data in order to generate an intermediate item of breathing information. A further step is directed to a comparison of the intermediate breathing information item with the item of training breathing information. A further step is directed to an adaptation of the trained function on the basis of the comparison. A further step is directed to a provision of the trained function.

According to some exemplary embodiments, the bioelectrical training signals and/or the training pressure loading signal is generated with a differential voltage measuring system according to exemplary embodiments according to the present invention and the item of training breathing information is generated in parallel thereto with a measuring system which is based upon a measurement method different from a differential voltage measurement with capacitive sensor elements, and in particular on a measuring system using a respiratory belt and/or a camera or radar-based measuring system.

According to one aspect, a method for providing an item of information indicating a breathing of a patient, in particular with a differential voltage measuring system according to exemplary embodiments of the invention is provided. The method has a plurality of steps. A first step is directed to a capture of a number of measurement signals relating to the patient with at least two capacitive sensor elements. A further step is directed to determining at least one bioelectrical signal from the measurement signals. A further step is directed to ascertaining the item of information indicating a breathing of the patient based upon the determined, at least one, bioelectrical signal.

The advantages and further developments disclosed regarding the exemplary embodiments of the differential voltage measuring system can be transferred to the above method. In particular, elements of the differential voltage measuring system and its embodiments can be converted into corresponding method steps.

According to some exemplary embodiments, the method further comprises the steps: capturing pressure signals, with at least one pressure-sensitive measuring element, indicating a pressure loading by the patient on an underlay and determining at least one, in particular spatially resolved, pressure loading signal from the pressure signals, wherein the step of ascertaining the item of information indicating a breathing of the patient additionally takes place based upon the pressure loading signal.

In a further aspect, embodiments of the present invention relate to a computer program product having a computer program which is directly loadable into a storage facility of a differential voltage measuring system, having program portions in order to carry out all the steps of the method according to the above exemplary embodiments when the computer program is executed in the differential voltage measuring system.

Embodiments of the present invention relate in a further aspect to a computer-readable storage medium on which program portions that can be read in and executed by a computer unit are stored, in order to carry out all the steps of the method according to the above exemplary embodiments when the program portions are executed by the computer unit.

The computer program products can therein comprise an item of software with a source code which must still be compiled and linked or which must only be interpreted, or an executable software code which, for execution, must only be loaded into the computer unit. With the computer program products, the inventive method can be carried out rapidly, exactly reproducibly and robustly. The computer program products are configured so that they can carry out the method steps according to embodiments of the present invention by the computer unit. The computer unit must have the respective pre-conditions such as, for example, a suitable working memory store, a suitable processor, a suitable graphics card or a suitable logic unit so that the respective method steps can be carried out efficiently.

The computer program products are stored, for example, on a computer-readable storage medium or are deposited on a network or server from where they can be loaded into the processor of the respective computer unit, which can be configured directly connected to the computer unit, or which can be configured as part of the computer unit. Furthermore, control information of the computer program products can be stored on a computer-readable storage medium. The items of control information of the computer-readable storage medium can be configured such that they carry out a method according to embodiments of the present invention when the data carrier is used in a computer unit. Examples of computer-readable storage media are a DVD, a magnetic tape or a USB stick, on which electronically readable control information, in particular software, is stored. If these items of control information are read from the data carrier and stored in a computer unit, all the embodiments/aspects according to the present invention of the above-described methods can be carried out. Embodiments of the present invention can therefore also proceed from the aforementioned computer-readable medium and/or the aforementioned computer-readable storage medium. The advantages of the proposed computer program products and/or of the associated computer-readable media substantially correspond to the advantages of the proposed methods.

### BRIEF DESCRIPTION OF THE DRAWINGS

Further special features and advantages of the present invention are apparent from the following description of exemplary embodiments, making reference to schematic drawings. Modifications mentioned in this regard can each be combined with one another in order to form new embodiments. In the different figures, the same reference signs are used for the same features.

In the figures:

FIG. 1 is a view of a differential voltage measuring system according to an exemplary embodiment;

FIG. 2 is a view of a differential voltage measuring system with signal measuring circuits according to a further exemplary embodiment;

FIG. 3 is an exemplary signal processing module of a signal processing apparatus according to an exemplary embodiment;

FIG. 4 is a view of a differential voltage measuring system according to a further exemplary embodiment;

FIG. 5 is a view of a sensor element according to an exemplary embodiment;

FIG. 6 is a view of a sensor element according to a further exemplary embodiment;

FIG. 7 is a flow diagram of a method for determining a breathing activity of a patient according to an exemplary embodiment;

FIG. 8 is signal paths associated with a method or a system for determining a breathing activity of a patient according to an exemplary embodiment;

FIG. 9 is a view of a trained function according to an exemplary embodiment; and

FIG. 10 is a flow diagram of a method for providing a trained function according to an exemplary embodiment.

### DETAILED DESCRIPTION

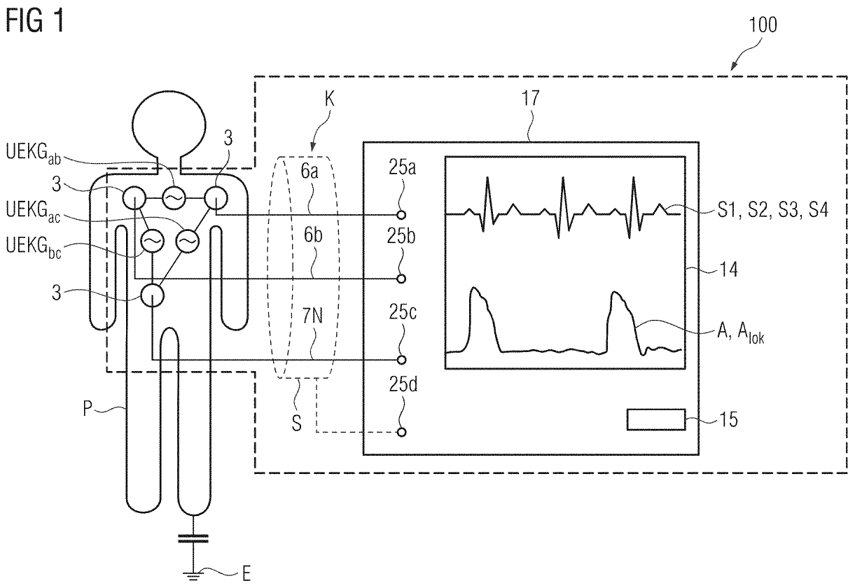

FIG. 1 shows a view of a differential voltage measuring system 100 arranged on a patient P. The voltage measuring system 100 comprises a combined ECG and breathing detection device 17 with its electrical terminals and sensor elements 3 connected thereto via cables K in order to ascertain bioelectrical signals S1, S2, S3, S4 on the patient P. At least one, preferably all the sensor electrodes 3 can be configured as part of a voltage measuring system 100 according to embodiments of the present invention as described in relation to other figures.

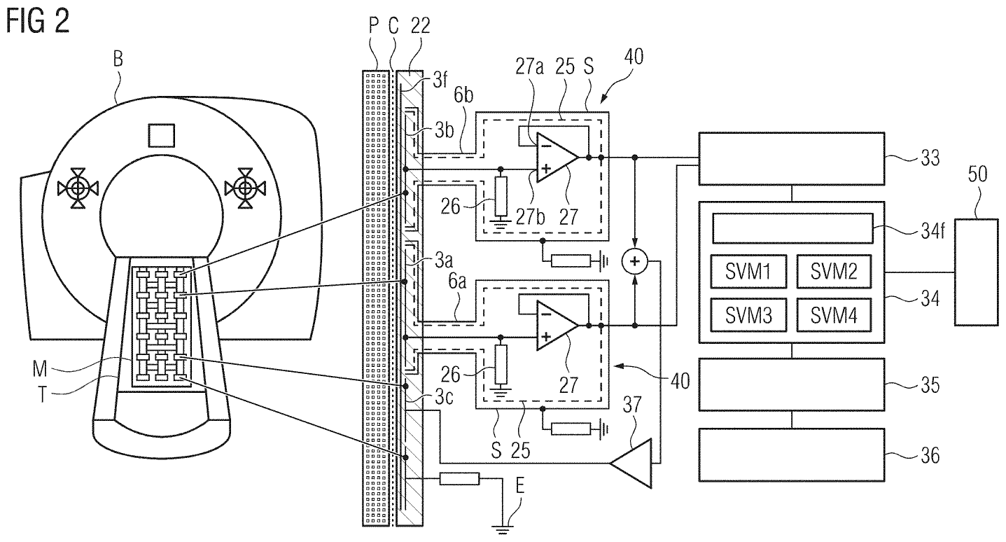

In order to ascertain the bioelectric signals S1, S2, S3, S4, at least one first sensor element 3 and one second sensor element 3 are needed, each having a sensor electrode 3a, 3b (see FIG. 2) which are mounted at, on or under the patient P. By way of signal measuring cables K, the sensor electrodes 3a, 3b are connected via terminals 25a, 25b, usually plug-in connectors, to the device 17. The first electrode 3a and the second electrode 3b together with the signal measuring cables K therein form a part of a signal capture unit, with which the bioelectrical signals S1, S2, S3, S4 can be captured.

A third sensor element 3 has a third sensor electrode 3c which serves as a reference electrode in order to create a potential equalization between the patient P and the device 17. Classically, this third electrode 3c is attached to the right leg of the patient (as a right leg drive or RLD). However, it can also be positioned at a different site. Furthermore, using further terminals (not shown), a plurality of further contacts can be mounted on the device 17 for further terminal leads (potential measurements) on the patient P and used for the formation of suitable signals.

The voltage potentials UEKGab, UEKGbc and UEKGac form between the individual electrodes 3a, 3b, 3c and are used for measuring the bioelectrical signals S1, S2, S3, S4.

The bioelectrical signals S1, S2, S3, S4 ascertained can be displayed on a user interface 14 of the device 17. These can comprise, for example, an ECG signal, an ECG vector or a muscle activity. In addition, on the user interface 14, breathing activity signals and/or breathing information A, Alok which are derived from the bioelectrical signals S1, S2, S3, S4 and optionally further measurement variables can be displayed. An item of breathing information A, Alok can comprise, for example, an item of information A regarding inhalation/exhalation cycles of the patient P or an item of information Alok regarding local breathing components which provides local breathing components regarding how the breathing has affected different body parts.

During the measurement, the patient P is coupled at least capacitively to the ground potential E (represented by a coupling to the right leg).

The signal measuring cables K which lead from the first sensor electrode 3a and the second sensor electrode 3b to the device 17, are a portion of the useful signal paths 6a, 6b and/or the corresponding sensor lines. The signal measuring cable K which leads from the electrode 3c to the device 17 herein corresponds to a portion of a third useful signal path 7N. The third useful signal path 7N can serve, in particular, to transfer interference signals that have been coupled in via the patient P and the electrodes.

The cables K have a screening S which is represented here schematically as a dashed cylinder surrounding all the useful signal paths 6a, 6b, 7N. The screening does not however have to surround all the cables K together, rather the cables K can also be separately screened. However, the terminals 25a, 25b, 25c preferably each have a pole integrated for the screening S. These poles are then brought together to a common screening terminal 25d. The screening S is configured therein, for example, as a metal foil surrounding the conductor of the respective cable K, which however, is insulated from the conductor.

In addition, the device 17 can have an external interface 15 in order to provide, for example, a connection for a printer, a storage facility and/or even a network. The device 17 has signal measuring circuits 40 associated with each of the terminals 25a, 25b (see e.g. FIG. 2) according to exemplary embodiments of the present invention. The signal measuring circuits 40 are each themselves connected via a ground switch 31 to ground E.

FIG. 2 shows a view of an exemplary differential voltage measuring system 100, comprising two signal measuring circuits 40 according to an exemplary embodiment. The two signal measuring circuits 40 have an identical structure, so that for the sake of clarity, corresponding components have largely been provided with reference signs only once.

The arrangement of a single sensor 3 and/or a single sensor electrode 3a of the signal measuring circuit 40 is illustrated here in the form of a capacitive measuring circuit. The patient P and the sensor electrode 3a are situated in spatial proximity to one another. Stated more precisely, the patient P lies here on a patient table T of an imaging system B in the form of a computed tomography system. A capacitive patient mat M, on which the patient P is positioned, is arranged on the patient table T. The patient mat M comprises a plurality of signal measuring circuits 40 according to embodiments of the present invention. The mat M can alternatively be configured as an electrode pad which can be arranged, in particular, in a backrest of an examination or treatment chair. Two of the signal measuring circuits 40 are described in greater detail below. The patient P can be provided, for example, with a textile covering C. Optionally lying thereupon is a cover 22 which is transparent to X-rays. The sensor electrodes 3a, 3b, 3c are not in direct electrical contact with the patient P, but are electrically insulated from the patient P at least by a sensor covering 3f. However, a capacitive decoupling of a signal is not impaired by the sensor cover 3f. The sensor electrode 3a, 3b, 3c, a sensor line 6a extending from the sensor electrode 3a, 3b, 3c to an operational amplifier 27, and the measuring circuit 40 comprising the operational amplifier 27 are surrounded by a so-called active protective screen 25 and preferably a screening S.

The operational amplifier 27 is configured as a so-called follower. I.e. the negative input 27a of the operational amplifier 27 is coupled to the output 28 of the operational amplifier 27. In this way, a high virtual input impedance is achieved for the operational amplifier 27 at the positive input 27b. This means that due to the voltage adaptation between the output 28 and the positive input 27b, hardly any current flows between the sensor element 3 and the active protective screen 25. Furthermore, the positive input 27b of the operational amplifier 27 is maintained, with the aid of a resistor 26 connected to the measuring device ground (also called “measuring ground”), at an electrical bias voltage. Thereby, the positive input can be set to a desired measurement potential. In this way, DC components are suppressed. This is desirable since the sensor electrode 3a, 3b, 3c should couple, above all, capacitively and a changing potential should be prevented.

The measurement signal MS stored in the corresponding amplifier circuits is digitized by an AD converter (not shown) and is transferred by way of a switching matrix 33 to a signal processing apparatus 34.

The signal measuring circuits 40 shown each comprise a sensor element 3 which, as shown, for example, in FIG. 5 can be designed with a mechanical mounting 10. The active guard 25 and the shield S each enclose the sensor electrode 3 in order to screen it effectively. The active guard 25 and the shield S further surround the sensor line 6a and, together with it penetrate the mechanical mounting 10, on the route to the operational amplifier 27. Alternative arrangements of the sensor line 6a are naturally also conceivable. The signal measuring circuits 40 therefore provide capacitive measuring signals MS which are modulated, in particular, by an ECG activity of the patient P. According to some embodiments, the measurement signals MS can therefore also be referred to as ECG measurement signals.

A further electrode is also provided in the patient mat M shown here for at least capacitive coupling of the patient to the ground potential E.

A further electrode and/or the associated measuring circuit 37 functions in the patient mat M as a reference electrode and/or as a so-called driven neutral electrode (DNE).

The differential voltage measuring system 100 further comprises a switching apparatus in the form of a switch matrix 33. In the presence of a large number of sensor electrodes 3, it serves to select which measurement signals MS of the sensor electrodes are used for a further signal processing.

The differential voltage measuring system 100 further comprises a signal processing apparatus 34 in the form of a so-called signal processing box, for instance. This is designed to carry out a pre-processing of the captured measurement signals MS in order to remove interference components and/or suitably to filter and/or combine the measurement signals MS. The signal processing apparatus 34 can be configured to carry out a processing with frequency-based prefilters 34f such as band-pass or band-stop filters, high pass, low pass or comb filters, but also an extended interference suppression such as, for example, in the German patent application DE 102019203627A. The prefilter 34f can have different filter modules in order to filter out suitable signal components and to suppress undesirable signal components, dependent upon the bioelectrical signals S1, S2, S3, S4 to be derived from the measurement signals MS. The filter modules can be realized by way of physical modules and/or virtual, in particular software-based, modules.

The signal processing apparatus 34 is configured, in particular, to derive one or more bioelectrical signals S1, S2, S3, S4 from the input variables comprising the measurement signals MS. For the calculation of the bioelectrical signals S1, S2, S3, S4, the signal processing apparatus 34 can have respective signal processing modules SVM1, SVM2, SVM3, SVM4. The subdivision into signal processing modules SVM1, SVM2, SVM3, SVM4 that has been undertaken therein serves merely for simpler explanation of the functioning of the signal processing apparatus 34 and is not to be understood as restrictive. The signal processing modules SVM1, SVM2, SVM3, SVM4 and their functions can also be grouped together in one element. The signal processing modules SVM1, SVM2, SVM3, SVM4 can therein also be understood as computer program products or computer program segments which, in the embodiment in the signal processing apparatus 34, realize one or more of the functions or method steps described below.

According to one embodiment, the bioelectrical signals S1, S2 comprise ECG signals of the patient P. For this purpose, in particular, frequencies of between 1 and 40 Hz are relevant. Therefore, the prefilter 34f can have a corresponding filter module which filters such frequencies from the measurement signals MS and passes them on. A filter module of this type can be configured, for example, as a digital bandpass and notch filter.

On the basis of such a prepreparation, for example, a beat-to-beat heartbeat of the patient P can be derived as a bioelectrical signal S1. For this purpose, the signal processing apparatus 34 can have a heartbeat signal processing module SVM1 which is designed to recognize a heartbeat of the patient P, in particular, in the preprocessed measurement signals MS. With regard to a subsequent breathing detection, this is based upon the recognition that on inhalation, the heart of the patient P beats faster than during exhalation.

Furthermore, an ECG vector signal S2 can be ascertained from the measurement signals MS preprocessed in particular by filtration in the range of 1 to 40 Hz. For this purpose, measurement signals MS of a plurality of sensor elements 3 are evaluated in a corresponding ECG vector signal processing module SVM2 of the signal processing apparatus 34. This is based upon the recognition that the heart rotates in the body during breathing, so that the location-dependent ECG signals measured from outside also change. In other words, an ECG vector combined from a plurality of signals rotates. Expressed in simplified form, the amplitude of the ECG signals rotates between the sensor elements 3.

According to further examples, a bioelectrical signal S3 which is modulated by a muscle activity of the patient can be obtained from the measurement signal MS. This is based upon the recognition that particularly with a plurality of sensor elements 3, it is highly probable that one or more sensor elements 3 contact the ribcage of the patient P in a region in which the muscle activity is measurable during the breathing of the patient P. Since for this purpose, signal components of 10 Hz to 100 Hz are relevant, the prefilter 34f can have a corresponding filter module which can be configured, for example, as a digital bandpass and notch filter. The signal processing apparatus 34 can accordingly have a muscle activity signal processing module SVM3 which is configured, on the basis of the measurement signals MS possibly suitably preprocessed by the prefilter 34f, to determine a muscle activity of the patient.

According to further examples, a bioelectrical signal S4 which is modulated by the capacitive coupling of the patient P to the sensor elements 3 and/or indicates such a coupling can be obtained from the measurement signals MS. The mapping of electric fields on the measurement signal tapped off at the sensor element 3 substantially depends, in the case of differential voltage measurement with capacitive sensors, upon the contact pressure from the patient P on the sensor element 3. This pressure changes during the breathing by the weight displacement of the body of the patient P. In principle, a measurement based upon such a change in the electric fields would permit a conclusion to be drawn regarding the breathing activity of the patient. A difficulty therein lies, however, in the fact that the electric fields can change for other reasons and it is possible ad hoc only with difficulty to make a statement regarding whether a corresponding measurement variable is based upon a breathing movement of the patient P or other extrinsic or intrinsic effects of the measuring system 100. Thus, field effects induced by the breathing movement can be masked and/or overlaid by changes of field variables imposed from outside. According to the present embodiment, it is therefore provided to place the change in the electric fields at individual sensor elements 3 in relation to a reference signal RS so that capacitive coupling can be inferred. As signals that are always available in an electrically powered environment, the electric fields of the mains power supply at 50 Hz and/or 60 Hz and their harmonics suggest themselves as a reference signal RS. It is therefore not necessary to apply a separate test signal.

The reference signal RS can be fed in by a reference signal capture apparatus 50. The reference signal capture apparatus 50 can comprise, for example, a measuring arrangement for measuring a screening current flowing on the screening S of the capacitive sensors 3, 4, said screening current flowing between the screen S and a GND potential connected to the individual sensors 3. Each screening of each sensor element 3 can be measured individually, and also a screening current can be measured via a common measuring point that is connected to GND. The screening of the cable connection 6a, 6b of the sensor elements 3 to the amplifier circuits of the capacitive sensor elements 3 can also be used for the measurement. In addition, the measuring point can also lie between the amplifier circuits and the units of the measuring system connected thereto. Furthermore, the reference signal capture apparatus 50 can comprise a driver circuit for the right leg. With a driver circuit of this type for the right leg, an interference acting directly on the patient due to an electric field can be ascertained by measuring a so-called RLD current and can be taken into account for determining the reference signal RS. In addition or alternatively to the aforementioned, the reference signal capture apparatus 50 can also comprise a ground current measuring unit which is configured to measure a current (ground current) which flows from the patient side in the direction of the measuring system side. The measuring point can lie between the GND points of the measuring side and the GND point of the further processing circuit of the measuring system. The GND point of the further processing circuit can be earth; the measuring side may not and cannot be at the earth potential for this use.

The measured currents, that is the RLD current, the screening current and the earth current which each correlate with different components of the measurement signal MS are used either individually or in combination as a reference signal RS for an analysis of the measurement signal MS in order to obtain information regarding a local capacitive coupling and thus a breathing movement of the patient P. For this purpose, the relevant frequency components at 50 Hz and/or 60 Hz and the harmonics thereof are firstly extracted from the reference signal RS and the measurement signals MS. For this purpose, the prefilter 34f can have a suitable filter module, for instance in the form of a comb filter. The signals processed in this way are then input into a comparator signal processing module SVM4 or a comparator module SVM4. The comparator signal processing module SVM4 is designed, on the basis of the possibly preprocessed measurement signals MS and the reference signal RS, to determine corrected measurement signals as bioelectric signals S4. In other words, the comparator module SVM4 is designed to filter the measurement signals MS on the basis of the reference signal RS and thereby to calculate out from the measurement signals MS the amount of field changes that do not originate from a local change in the contact pressure from the patient P on the sensor element 3.

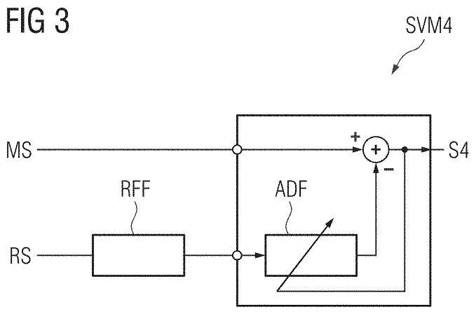

FIG. 3 shows, by way of example, a possible embodiment of the comparator module SVM4. The comparator module SVM4 comprises an adaptive filter ADF and a reference formation filter RFF. The comparator module SVM4 is configured, in particular, to generate a corrected measurement signal as a bioelectrical signal S4 on the basis of a digitized measurement signal MS and a digitized reference signal RS. The adaptive filter ADF is designed to estimate a transfer function which gives a relationship between the measured reference signal RS and the modulation coupled into the measurement signal MS by a breathing movement. With the aid of the transfer function, the adaptive filter ADF generates an estimated subtraction signal which can be subtracted from the measurement signal MS. Furthermore, the comparator module SVM4 has the property of being able to amend independently its transfer function and therewith the estimated subtraction signal and its frequency in operation. Therein, dependent upon an output signal of the compensation module SVM4, an error signal is generated and the coefficients of the transfer function generated by the adaptive filter unit ADF and applied are amended dependent upon the error signal such that the error signal is minimized. The comparator module SVM4 finally outputs a bioelectrical signal S4 which is freed from interference effects by electric fields in the frequency range selected by the comb filter (50 Hz and/or 60 Hz and their harmonics) and thus enables a conclusion to be drawn regarding the capacitive coupling.

The comparator module SVM4 further comprises a reference formation filter RFF. This reference formation filter RFF is advantageous since the measured reference signals RS can deviate strongly from the measurement currents with regard to their frequency response and their phase and therefore otherwise, if it is at all possible, can only be subtracted with a great effort by an adaptive filter. The reference formation filter RFF is designed, for example, as a low pass filter which already has a damping effect above a few Hz and at 20 to 30 Hz reaches a damping of 25 to 30 dB. The reference formation filter RFF can preferably be realized digitally, i.e. configured as a virtual filter in the form of software, although it can also be realized as hardware.

The bioelectrical signals S1, S2, S3, S4 and the embodiments of the signal processing apparatus 34 related thereto should be understood as exemplary. Thus the signal processing apparatus 34 can be designed to provide only one of the bioelectrical signals S1, S2, S3, S4. Alternatively, the signal processing apparatus 34 can be designed to provide all the aforementioned bioelectrical signals S1, S2, S3, S4 or any desired subset thereof. In addition, the signal processing apparatus 34 can be designed to provide further and/or alternative bioelectrical signals which deviate from the bioelectrical signals S1, S2, S3, S4 named by way of example.

The bioelectrical signals S1, S2, S3, S4 ascertained by the signal processing apparatus 34 are input into the computer unit 35. The computer unit 35 is configured, on the basis of individual bioelectrical signals S1, S2, S3, S4 or combinations of the input bioelectrical signals S1, S2, S3, S4 to extract and/or derive an item of breathing information A, Alok. In particular, the computer unit 35 can be configured to bring corresponding computer program products or computer program segments into effect and to provide the item of breathing information A, Alok on the basis of the input variables that are available. According to some embodiments, the computer unit 35 can implement a model-based calculation, e.g. based upon a correspondingly configured Kalman filter. According to other embodiments, the computer unit 35 can implement a trained function TF which has been adapted to this task with methods of machine learning.

Furthermore, the differential voltage measuring system 100 can comprise a trigger apparatus 36. This can be configured to recognize breathing cycles of the patient P from the item of breathing information A, Alok, in order therefrom to generate control signals comprising a trigger and/or start time point information item for a medical imaging system B. The trigger apparatus 36 can further be configured to recognize a heartbeat of the patient P from the bioelectrical signals S1, S2, S3, S4, in order therefrom to generate control signals comprising an item of trigger and/or start time point information for a medical imaging system B. On the basis of the control signals of the trigger apparatus 35, an imaging system B can then ascertain, for example, time points for an image acquisition.

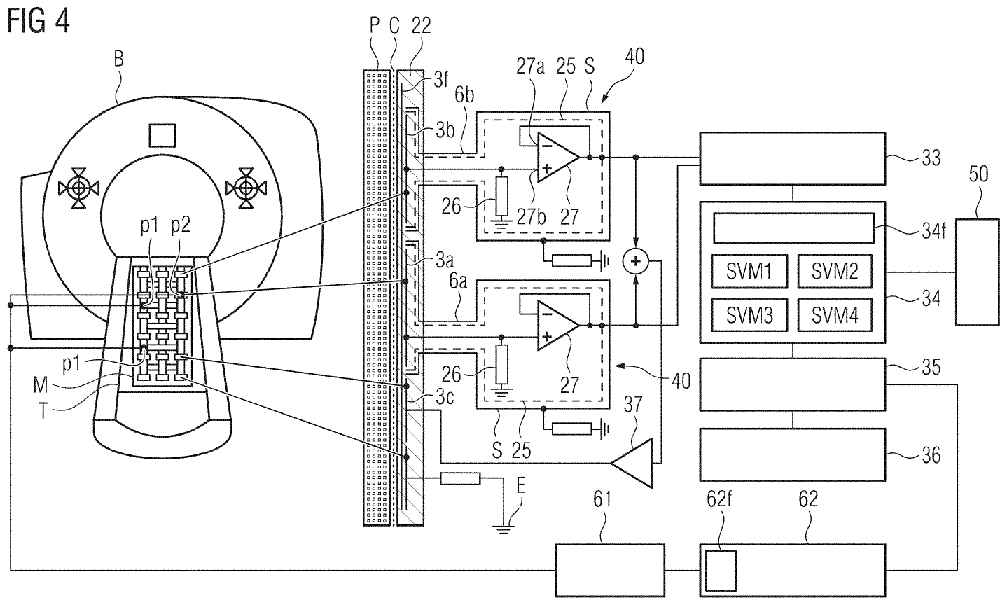

FIG. 4 shows schematically a further exemplary embodiment of the voltage measuring system 100 according to the present invention. The same reference signs as in the previous embodiments therein denote the same or functionally similar components.

The embodiment shown in FIG. 4 differs from the previous embodiment in that the patient mat M additionally has pressure sensors p1, p2 which capture a local pressure signal DS at the sensor location and provide the processing as an additional information source. This is based upon the consideration that the weight of the patient P becomes displaced during breathing, so that the pressure sensors p1, p2 are stimulated. If a plurality of pressure sensors p are included at different sites in the patient mat M, the pressure sensors p1, p2 are stimulated to different degrees depending upon the arrangement position in the patient mat M and from the pressure signals DS there arises a locally resolved image of the pressure loading of the patient mat M by the patient P. The pressure sensors p1, p2 can be mounted at a suitable position for breathing detection. Such a position is, for example, in the middle of the patient mat M under the spine of the patient P upon which the patient does not lie on a sustained basis and, due to the volume change during the breathing, a different spacing results between the patient P and the patient mat M. A pressure sensor p1, p2 installed protruding in relation to the remainder of the patient mat M would be strongly stimulated by this spacing change.

The pressure signals DS ascertained with the pressure sensors p1, p2 are input via signal measuring cables K via suitable connections, usually plug-in connections, into a secondary signal processing apparatus 62. The first pressure sensors p including the signal measuring cables K therein form a part of a secondary useful signal path with which the pressure signals DS of the pressure sensors p1, p2 can be captured. The pressure measurement signals DS can therein be digitized by an AD converter (not shown) before input into the secondary signal processing apparatus 62. The secondary signal processing apparatus 62 is configured to extract an item of information from the pressure signals DS which indicates a, in particular local, pressure loading by the patient P. A switching apparatus 61 can optionally be connected upstream of the secondary signal processing apparatus 62, said switching apparatus being able, similarly to the switch matrix 33, to select which of the pressure sensors p1, p2 are used for a further signal processing. Furthermore, the secondary signal processing apparatus 62 can have a prefilter 62f which filters the incoming pressure signals DS according to suitable frequencies. The frequency range can therein lie between 0.05 Hz and 5 Hz. The prefilter 62f can be realized with physical, i.e. hardware-based, modules and/or virtual, in particular software-based, modules.

In the secondary signal processing apparatus 62, the pressure signals DS are processed to a signal SN which indicates a pressure loading of the patient mat M by the patient P (hereinafter also named the pressure loading signal). This signal SN is then input into the computer unit 35 which accordingly thereto is configured additionally to take account of the signal SN in the breathing detection, i.e. the determination of the item of breathing information A, Alok. According to some embodiments, the computer unit 35 can also be configured such that it determines the item of breathing information A, Alok purely on the basis of the signal SN, that is without taking account of the bioelectrical signals S1, S2, S3, S4.

According to embodiments of the present invention, two different types of pressure sensors p1, p2 are conceivable. Firstly, separate pressure sensors p1 can be included in the patient mat M, designed differently from the sensor elements 3 for differential voltage measurement. For this, pressure sensors known from the prior art can be used.

The combination of the pressure measurement with the ascertainment of the bioelectrical signals S1, S2, S3, S4 also opens up the possibility of integrating pressure sensors as integrated pressure sensors p2 in the sensor construction of the capacitive sensor elements 3. Therein, the integrated pressure sensors p2 can be integrated, in particular, into a mechanical mounting 10 for the sensor electrode 3a.

An embodiment for such a mechanical mounting 10 is shown in FIG. 5. The mechanical mounting 10 is configured to be at least partially compressible, that is, at least in some regions, i.e. it can be compressed. The mechanical mounting 10 is fastened to a substrate U of the sensor element construction and supports the sensor electrode 3 against the substrate U. The substrate U is formed, for example, by the underside/the support surface of a capacitive patient mat M. As a direct support element for the sensor electrode 3, the mechanical mounting 10 comprises a supporting structure 5. The supporting structure 5 is arranged beneath the sensor electrode and has a base area corresponding at least to the base area of the sensor electrode 3, so that the sensor electrode 3 lies completely on the supporting structure 5. The mechanical mounting 10 comprises as a further component, a carrier structure 7. This is arranged beneath the supporting structure 5 and above the substrate U. The supporting structure 5 is formed, in particular, of a compressible foam material. The carrier structure 7 covers the substrate in this embodiment in a full-surface manner over the base area of the measuring system 1. In this embodiment, the carrier structure 7 is also formed from a compressible foam material. The carrier structure 7 is therein designed to be, in particular, harder than the supporting structure 5. In the present case, the carrier structure 7 is designed to be 25% harder than the supporting structure 5. In particular, in embodiments in which the sensor element 3 is integrated plurally, for example, into a capacitive patient mat, the carrier structure enables an equalization of a different height level of the patient surface. For example, by the carrier structure 7, a lordosis form can be compensated for so that despite the variable height level, all the individual measuring systems 1 of the patient mat M can provide a measurement signal MS. In lordosis, the carrier structure 7 would hardly be compressed, but with patents P having a straight back, correspondingly more.

Herein, the base area of the supporting structure 5 is, for example, square with the dimensions 3 cm×3 cm in accordance with the base area of the sensor electrode 3a. The height of the supporting structure 5 is, for example, 8 cm. In the present case, the supporting structure consists of a viscoelastic foam material with a high degree of energy absorption, in particular, the foam material GV 50/30. This is distinguished in that it generates a substantially constant counterforce for small compression changes.

In order to achieve a particularly even force input over the base area of the measuring system 1 into the carrier structure 7, in this embodiment, the mechanical mounting 10 optionally comprises an intermediate layer 9. This connects the carrier structure 7 to the supporting structure 5. This means it is also formed in a full-surface manner above the carrier structure 7, at least over the base area of the measuring system 1. The optional intermediate layer 9 provides, in particular, for a distribution of movement-related, highly locally very restrictively acting force peaks onto further regions of the carrier structure, whereby an inclination and/or tilting of the supporting structure 5 and/or the sensor electrode 3a remains minimized and thereby the sensor surface always remains oriented substantially plane-parallel to the patient surface in order to ensure an optimum signal tap-off.

In order to provide the pressure signals DS, the carrier structure 7 is equipped as the measuring element and/or the integrated pressure sensor p2. For this purpose, for example, a foam material of the carrier structure 7 can be configured conductive in such a way that on compression its conductivity changes. By way of a continuous measurement of the current bulk resistance, the current compression can thereby be captured. This measurement can take place by way of two conductive layers, one directly above and one beneath the carrier structure 7, which have a constant current applied to them from the secondary useful signal path, and the secondary useful signal path captures the voltage drop caused thereby and passes it on to the secondary signal processing apparatus 62 as a pressure measurement signal DS.

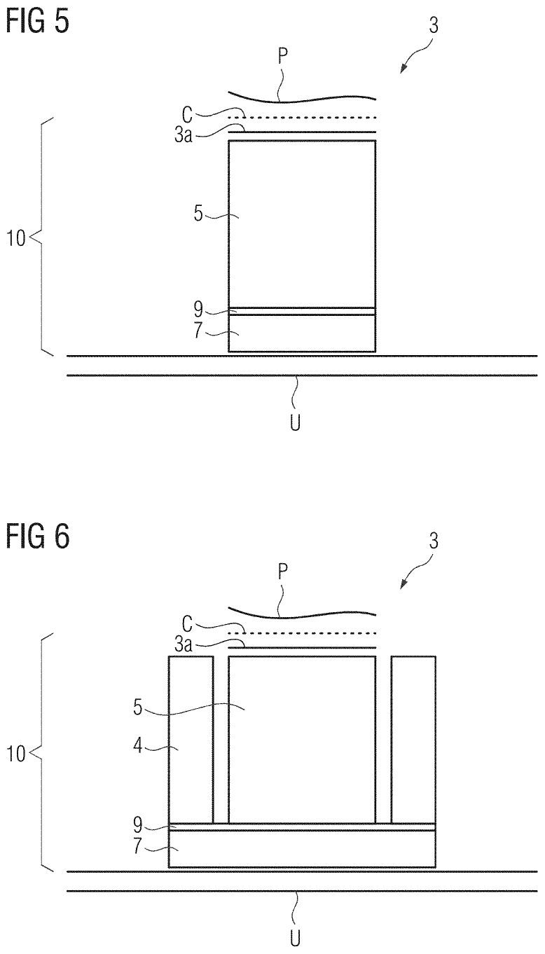

According to an alternative embodiment shown in FIG. 6, the mechanical mounting 10 further comprises a frame structure 4. The frame structure 4 at least partially surrounds the supporting structure 5. In this embodiment, the frame structure 4 is provided on two sides of the supporting structure 5. In alternative embodiments, the frame structure 5 can be a structure that is closed round the supporting structure 5. The supporting structure 5 is constructed to be higher than the frame structure 4. Thus, in the unloaded state, it extends further from the substrate U in the direction of the patient P than the frame structure 4.

The frame structure 4 can be formed of a compressible material, in particular a foam material (which promotes patient comfort). It can however also consist of an incompressible material, for example, a plastics material or wood. In any event, the supporting structure 5 has a lower hardness than the frame structure 4. The supporting structure 5 consequently has a greater compressibility than the frame structure 4. The frame structure 4 must in any event be selected to be so firm that it can hold the supporting structure 5 constantly and in the relatively long term substantially at the height of the frame structure 4. By way of a force component FKomp of the weight force FBody of the patient P, the supporting structure 5 is now compressed to substantially the height of the frame structure 4. The predominant/remaining portion of the weight force (FBody−FKomp) is transmitted to the less compressible, or incompressible frame structure 4. The force component FKomp acting upon the sensor electrode 3a and/or the supporting structure 5 causes a counterforce FFoam generated by the supporting structure 5 in the direction of the patient P, which corresponds in quantity to the force component FKomp, thus, FKomp=−FFoam. This counterforce FFoam now remains constant even during patient movement, for example, due to shifting weight, or the heartbeat, since a maximum compression of the supporting structure 5 is preset by the frame structure 4. A variation of the weight force FBody acting has an effect, according to embodiments of the present invention, on the frame structure 4 only, and no longer on the supporting structure 5. This ensures, firstly, a derivation of the pressure loading by the patient on the carrier structure 7 in which the integrated pressure sensor p2 is arranged (see above). Secondly, a quality of the measurement of the bioelectrical signals S1, S2, S3, S4 is improved in this way since thereby interferences in the measurement signal MS are eliminated or at least reduced.

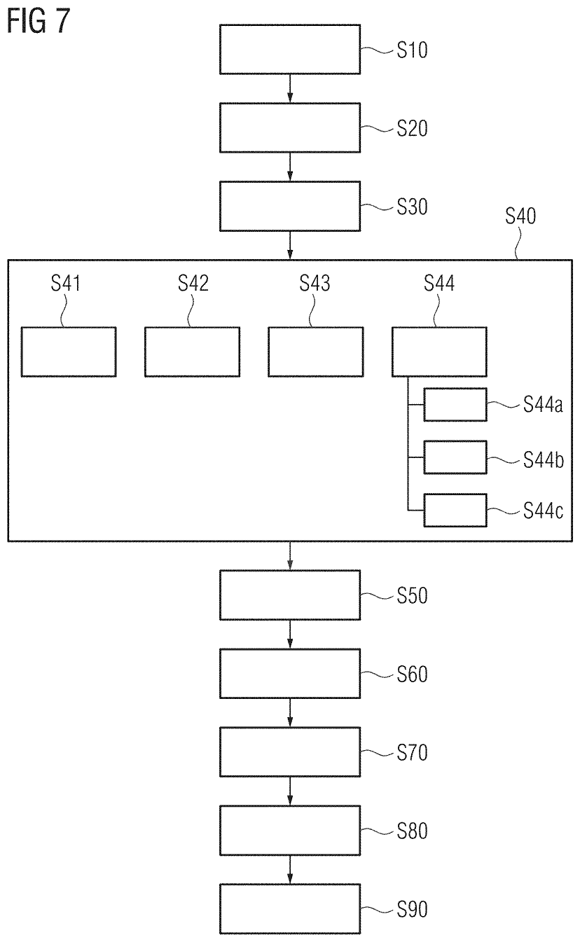

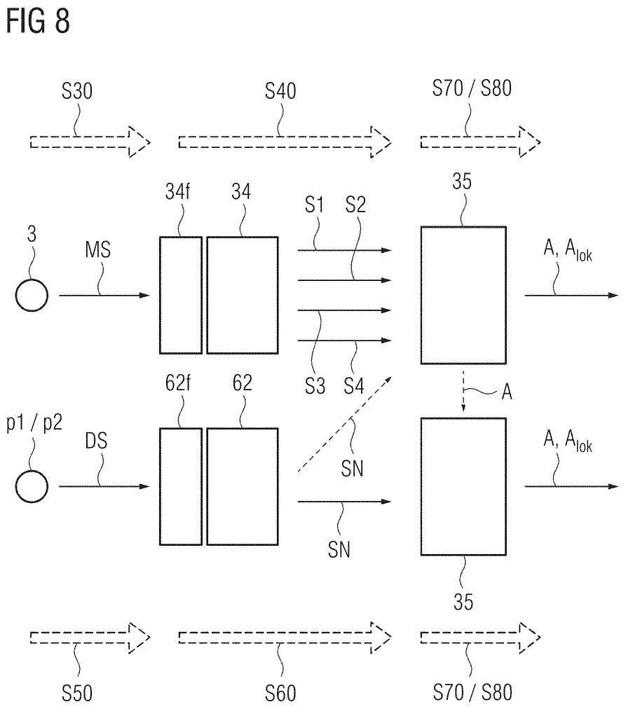

FIG. 7 shows a schematic flow diagram of a method for providing a breathing activity of a patient P. The sequence of the method steps is limited neither by the sequence shown nor by the selected numbering. Thus, the sequence of the steps can be exchanged if relevant and individual steps can be omitted. In addition, one or more steps, in particular, a sequence of steps and optionally the whole method can be carried out repeatedly. In FIG. 8, the data streams associated with the method shown in FIG. 7 are illustrated by way of example.

In a first step S10, a differential voltage measuring system according to one of the embodiments described here is provided. In a step S20, a patient P is arranged on the patient mat M.

Step S30 is directed to the capture of at least one measurement signal MS with at least one capacitive sensor electrode 3a, 3b, 3c. Step S30 can therein comprise a suitable preprocessing of the measurement signals MS, for instance a conversion of analogue measurement signals MS into digital measurement signals MS or a selection of suitable measurement signals MS from a plurality of measurement signals MS from different sensor electrodes 3a, 3b, 3c. Step S30 preferably takes place by the signal measuring circuit 40 and/or possibly the switching matrix 33 and possibly an AD converter integrated into the measuring path.

In step S40 a determination of one or more bioelectric signals S1, S2, S3, S4 takes place on the basis of the measurement signals MS, in particular via a digital signal processor. In addition, step S40 has the optional substeps S41-S44. Step S40 can comprise one or more of the substeps S41-S44. In step S41, a bioelectrical signal S1 which comprises a beat-to-beat heartbeat of the patient P is determined from the measurement signals MS captured and, if relevant, preprocessed in step S30. For this purpose, measurement signals MS can be suitably filtered in order to obtain relevant signal components which can lie, for example, in the range from 1 Hz to 40 Hz. In step S42, from the measurement signals MS captured and, if relevant, preprocessed in step S30 (preferably from a plurality of measurement signals MS), a bioelectrical signal S2 is determined which comprises an ECG vector of the patient P. Therein, in particular, the fact can be utilized that in the use of a plurality of different sensors 3 arranged in the patient mat M, a spatially resolved measurement of the measurement signals MS takes place, which enables the determination of an ECG spatial vector. Here also, relevant signal components lie in the range from 1 Hz to 40 Hz and a corresponding filtration step for the measurement signal MS can optionally be included in step S42. In step S43, from the measurement signals MS captured and possibly preprocessed in step S30, a bioelectrical signal S2 is determined which comprises a muscle activity of the patient P. Relevant signal components lie therein in the range from 10 Hz to 100 Hz. A corresponding filter step is optionally contained in step S43.

In step S44, from the measurement signals MS captured and possibly preprocessed in step S30, a bioelectrical signal S4 is determined which indicates a capacitive coupling of the patient P with the respective sensor electrode 3a, 3b, 3c and so permits a conclusion to be drawn regarding the breathing activity of the patient P. For this purpose, the measurement signals MS are filtered with a reference signal RS in order to compensate for variations in the electric fields. Accordingly, in a substep S44a, a capture of the reference signal RS with the reference signal capture apparatus 50 takes place. In a further substep S44b, the relevant frequency components at 50 Hz and/or 60 Hz and the harmonics thereof are firstly extracted from the reference signal RS and the measurement signals MS. For this purpose, in particular, a comb filter can be used. In a further substep S44c, the determination of a corrected measurement signal as a bioelectrical signal S4 takes place by way of filtration of the measurement signals MS on the basis of the reference signal RS.

Step S40 including possible substeps takes place by the signal processing apparatus 34 and, in particular using the modules SVM1, SVM2, SVM3, SVM4 and 34f applied therein.

Step S50 is directed to the capture of at least one pressure signal DS with the pressure sensors p1 and/or p2. For this, a signal which indicates the local pressure loading on the pressure sensors p1 and p2 can be tapped off with the secondary useful signal paths. In step S60, on the basis of these pressure signals DS, a signal SN is ascertained which represents an, in particular local, i.e. spatially resolved, pressure loading of the patient mat M by the patient P. This determination can take place by the secondary signal processing apparatus 62.

In step S70, the determination of the inhalations and exhalations of the patient P as an item of information A indicating the breathing of the patient P takes place on the basis of one or more of the bioelectrical signals S1, S2, S3, S4 and/or the pressure loading signal SN. In particular, step S70 can comprise an application of a Kalman filter and/or a trained function TF to the signals S1, S2, S3, S4 and/or SN. Step S70 can take place, in particular, by way of a correspondingly configured computer unit 35.

In the optional step S80, the inhalations and exhalations determined in step S70 can be used, while additionally taking account of the spatially resolved pressure loading signal SN as an item of information Alok indicating the breathing of the patient P, to determine a local breathing component which reveals how the inhalations and exhalations affect at least one body part of the patient P. Optionally, in step S80, one or more bioelectrical signals S1, S2, S3, S4 can again be taken into account separately—in particular the ECG vector signal S2 or the signal S4 inducing a (local) capacitive coupling, since these also contain locally resolved information. In particular, step S80 can comprise an application of a Kalman filter and/or a trained function TF to the inhalations and exhalations of the patient P determined in step S70 and the signals S1, S2, S3, S4 and/or SN. Step S80 can take place, in particular, by way of a correspondingly configured computer unit 35.

Finally, step S90 is directed to a provision of the determined inhalations and exhalations and/or the local breathing components, in particular, to a medical imaging system B in which imaging system this item of breathing information A, Alok can be used to optimize a capturing of medical image data and, in particular, to adapt it to the breathing of the patient P.

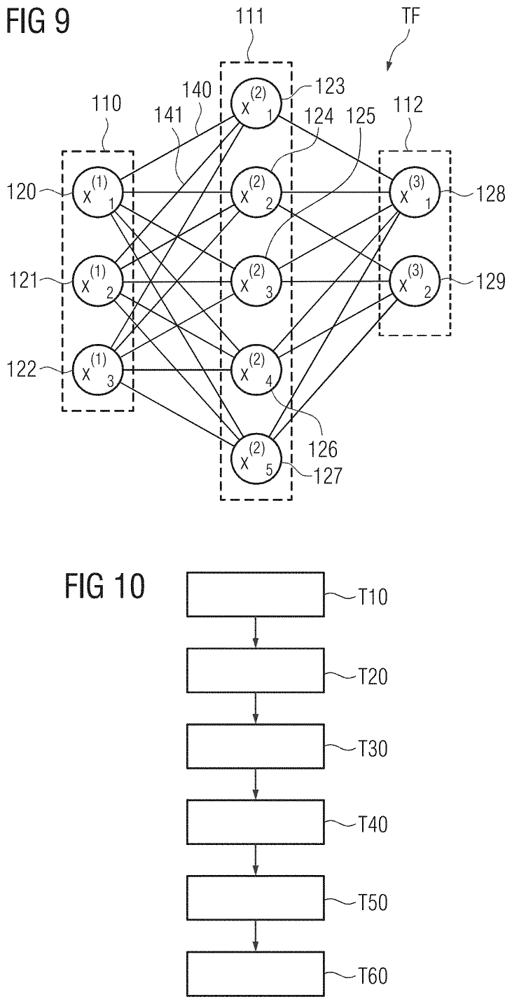

Represented schematically simplified in FIG. 9 is a trained function TF which is suitable for determining an item of breathing information A, Alok from the bioelectrical signals S1, S2, S3, S4 and/or from the pressure loading signal SN and/or can be trained for this. In the exemplary embodiment shown, the trained function TF is configured as a neural network. The neural network can also be designated an artificial neural net, artificial neural network or neural net.

The neural network 100 comprises nodes 120, . . . , 129 and edges 140, 141, each edge 140, 141 being a weighted connection of a first node 120, . . . , 129 to a second node 120, . . . , 129. In general, the first node 120, . . . , 129 and the second node 120, . . . , 129 are different nodes, but it is also possible that the first node 120, . . . , 129 and the second node 120, . . . , 129 are identical. An edge 140, 141 from a first node 120, . . . , 129 to a second node 120, . . . , 129 can also be designated an incoming edge for the second node and as an outgoing edge for the first node 120, . . . , 129.

The neural network 100 responds on input values x(1)1, x(1)2, x(1)3 to a plurality of input nodes 120, 121, 122 of the input layer 110. The incoming values x(1)1, x(1)2, x(1)3 are used in order to generate one or a plurality of outputs x(3)1, x(3)2. The node 120 is connected to the node 123 via an edge 140, for example. The node 121 is connected to the node 123 via the edge 141, for example.

In this exemplary embodiment, the neural network 100 learns by adapting the weighting factors wi, j (weights) of the individual nodes on the basis of training data. Possible input values x(1)1, x(1)2, x(1)3 of the input nodes 120, 121, 122 can be, for example, the bioelectrical signals S1, S2, S3, S4, the pressure loading signal SN and/or an item of information regarding inhalations and exhalations of the patient P.

The neural network 100 weights the input values of the input layer 110 on the basis of the learning process. The output values of the output layer 112 of the neural network 100 preferably correspond to an item of information A, Alok indicating the breathing of the patient P, such as the inhalations and exhalations of the patient or the local breathing components of the patient. The output can take place via a single, or a plurality of, output nodes x(3)1, x(3)2 in the output layer 112.

The artificial neural network 100 preferably comprises a hidden layer 111 which comprises a plurality of nodes x(2)1, x(2)2, x(2)3. A plurality of hidden layers can be provided, a hidden layer using output values of another hidden layer as input values. The nodes of a hidden layer 111 perform mathematical operations. An output value of a node x(2)1, x(2)2, x(2)3 therein corresponds to a non-linear function f of its input values x(1)1, x(1)2, x(1)3 and the weighting factors wi, j. After receipt of the input values x(1)1, x(1)2, x(1)3 a node x(2)1, x(2)2, x(2)3 performs a summation of a multiplication, weighted with the weighting factors wi, j, of each input value x(1)1, x(1)2, x(1)3, as defined by the following function:

xⱼ⁽ⁿ⁺¹⁾=f(Σᵢ xᵢ⁽ⁿ⁾·w_{i, j}^{(m, n)}).

The weighting factor wi, j can be, in particular, a real number lying, in particular, in the interval [−1; 1] or [0; 1]. The weighting factor w_{i, j}^{(m, n)} denotes the weight of the edge between the i-th node of an m-th layer 110, 111, 112 and the j-th node of the n-th layer 110, 111, 112.

In particular, an output value of a node x(2)1, x(2)2, x(2)3 is formed as a function f of a node activation, for example, a sigmoid function or a linear ramp function. The output values x(2)1, x(2)2, x(2)3 are transferred to the output node(s) 128, 129. A summation of a weighted multiplication of each output value x(2)1, x(2)2, x(2)3 is calculated anew as a function of the node activation f and thereby the output values x(3)1, x(3)2 calculated.

The neural network TF shown here is a feedforward neural network in which all the nodes 111 process the output values of a previous layer in the form of its weighted sum as input values. Self-evidently, according to embodiments of the present invention, other neural network types can be used, for example, feedback networks in which an input value of a node can simultaneously also be its output value.

The neural network TF can be trained by a method of supervised learning in order to provide the item of breathing information A, Alok. A known procedure is back-propagation which can be applied for all the exemplary embodiments of the present invention. During the training, the neural network TF is applied to training input data and/or values and must generate corresponding, previously known training output data and/or values. Mean square errors (MSE) between calculated and expected output values are calculated iteratively and individual weighting factors are adjusted until the deviation between the calculated and expected output values lies below a pre-determined threshold.

In order to provide training input data, bioelectric signals S1, S2, S3, S4 and/or pressure loading signals SN from a test person P situated on the patient mat M can therein be generated substantially in association with the previous embodiments, in particular, using a differential voltage measuring system 100. Suitable training output data can be generated particularly efficiently in that, in parallel, the breathing of the test person P is recorded in order to gather comparison data corresponding to the breathing information A, Alok which is ultimately to provide the trained function TF. This can take place via spirometry, for instance, using a spirometer. In this way, the inhalations and exhalations of the patient during the measurement of the bioelectric signals S1, S2, S3, S4 and/or the pressure loading signals SN can be captured with a spirometer. Alternatively or additionally, training output data can be generated, for example, by way of a camera-based or radar-based capture of the breathing of the test person P. In particular, thereby a local breathing component Alok of the test person P can be determined. In addition or alternatively, the test person P can be provided, during the measurement of the signals S1, S2, S3, S4, SN, with one or more respiratory sensing belts which effectively enable a determination of the item of breathing information.

FIG. 10 shows an exemplary embodiment of a computer-implemented method for providing a trained function TF. The sequence of the method steps is not limited either by the sequence shown or by the selected numbering. Thus, the sequence of the steps can be exchanged if relevant and individual steps can be omitted. In addition, one or more steps, in particular, a sequence of steps and optionally the whole method, can be carried out repeatedly.

In step T10, training input data is provided, the training input data comprising, in particular, one or more bioelectric training signals S1, S2, S3, S4 and/or a training pressure loading signal SN, which have been determined from a test person P, preferably by a differential voltage measuring system 100.

In step T20, training output data is provided, the training output data being related to the training input data and, in particular, comprising an item of breathing information A, Alok determined in parallel with the determination of the training input data. Therein, the training output data is captured, in particular by a measuring facility that is different from the differential voltage measuring system 100 and is based, in particular, on a different measuring principle.

In a step T30, the trained function TF is applied to the training input data in order to generate intermediate output data. In step T40, this intermediate output data is compared with the training output data, whereupon the trained function TF in step T50 is adapted on the basis of the comparison. This can take place, for example, on the basis of a cost functional which penalizes deviations of the item of breathing information A, Alok in the intermediate output data from that in the training output data. One or more parameters of the trained function TF can then be adapted, in particular, so that the cost functional is minimized, for example, by back propagation. In order to minimize the cost functional, the comparison is carried out for different paired sets from training output data and training output data as well as intermediate output data until a local minimum of the cost functional is achieved and the trained function TF works satisfactorily. In step T60, the thus adapted trained function TF is finally provided.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Where it has not yet explicitly been set out, although useful and in the spirit of the present invention, individual exemplary embodiments, individual sub-aspects or features thereof can be combined and/or exchanged with one another without departing from the scope of the present invention. Advantages of the present invention described in relation to an exemplary embodiment also apply, where transferable, to other exemplary embodiments without this being explicitly stated.

What is claimed is:

1. A differential voltage measuring system to determine a breathing activity of a patient, the differential voltage measuring system comprising:
a number of signal measuring circuits, each having a capacitive sensor element configured to capture a measurement signal relating to the patient;
a signal processing apparatus configured to determine at least one bioelectrical signal from the measurement signals; and
a computer unit configured to
ascertain an item of breathing information on the basis of the at least one bioelectrical signal, said item of breathing information indicating the breathing activity of the patient, and
provide the item of breathing information.

2. The differential voltage measuring system as claimed in claim 1, wherein
the signal processing apparatus is configured to determine at least two different bioelectrical signals from the measurement signals; and
the computer unit is configured to take into account each of the at least two different bioelectrical signals to ascertain the item of breathing information.

3. The differential voltage measuring system as claimed in claim 1, wherein the at least one bioelectrical signal includes at least one of a beat-to-beat heart rate of the patient, an ECG vector of the patient or a muscle activity signal of the patient.

4. The differential voltage measuring system as claimed in claim 1, wherein
the at least one bioelectrical signal indicates a capacitive coupling between at least one of the capacitive sensor elements and the patient; and
the signal processing apparatus includes
a reference signal capture apparatus configured to capture a reference signal, and
a comparator module configured to
ascertain, on the basis of the reference signal, a corrected measurement signal, and
provide, on the basis of the corrected measurement signal, as the at least one bioelectrical signal, a signal from which a capacitive coupling between at least one of the capacitive sensor elements and the patient is derivable.

5. The differential voltage measuring system as claimed in claim 4, wherein the comparator module comprises:
a reference formation filter configured to filter the reference signal; and
an adaptive filter unit configured to filter a measurement signal on the basis of the filtered reference signal, to ascertain the corrected measurement signal.

6. The differential voltage measuring system as claimed in claim 4, wherein
the signal processing apparatus includes a prefilter, in the form of a comb filter configured to extract at least one of frequencies of 50 Hz, 60 Hz or their harmonics from at least one of a measurement signal or the reference signal.

7. The differential voltage measuring system as claimed in claim 1, further comprising:
a number of pressure-sensitive measuring elements, each of which is configured to capture a pressure signal indicating a local pressure loading by the patient on an underlay;
a secondary signal processing apparatus configured to determine at least one pressure loading signal from the pressure signals; and
wherein the computer unit is configured to ascertain and provide the item of breathing information on the basis of the at least one pressure loading signal.

8. The differential voltage measuring system as claimed in claim 7, wherein:
at least one of the capacitive sensor elements has a mechanical mounting, said mechanical mounting being configured to support a sensor electrode of the corresponding capacitive sensor element; and
at least one of the pressure-sensitive measuring elements is accommodated in the mechanical mounting.

9. The differential voltage measuring system as claimed in claim 1, further comprising:
an output unit configured to output the item of breathing information and at least one of the at least one bioelectrical signal, the at least one of the at least one bioelectrical signal indicating at least a heartbeat of the patient.

10. The differential voltage measuring system as claimed in claim 1, wherein the computer unit comprises:
a Kalman filter configured to ascertain the item of breathing information on the basis of signals input into the computer unit, and
provide the item of breathing information.

11. The differential voltage measuring system as claimed in claim 1, wherein the computer unit is configured to
apply a trained function to signals input into the computer unit, said trained function being configured to ascertain the item of breathing information on the basis of the signals input into the computer unit, and
provide the item of breathing information.

12. A method comprising:
using a differential voltage measuring system as claimed in claim 1 to synchronize a medical imaging system with the breathing activity of the patient.

13. A computer-implemented method for providing an item of breathing information indicating a breathing activity of a patient, the method comprising:
capturing a number of measurement signals relating to the patient with at least two capacitive sensor elements;
determining at least one bioelectrical signal from the measurement signals; and
ascertaining the item of breathing information of the patient on the basis of the at least one bioelectrical signal.

14. The method as claimed in claim 13, further comprising:
capturing pressure signals, with at least one pressure-sensitive measuring element, indicating a pressure loading by the patient on an underlay; and
determining at least one pressure loading signal from the pressure signals; wherein
the ascertaining the item of breathing information of the patient is further based on the pressure loading signal.

15. A non-transitory computer-readable storage medium storing a computer program having program portions that carry out the method as claimed in claim 13 when the computer program is executed at a differential voltage measuring system.

16. The different voltage measuring system of claim 7, wherein the at least one pressure loading signal includes a spatially resolved pressure loading signal.

17. The method as claimed in claim 14, wherein the at least one pressure loading signal includes a spatially resolved pressure loading signal.

18. The differential voltage measuring system as claimed in claim 2, wherein the at least one bioelectrical signal includes at least one of a beat-to-beat heart rate of the patient, an ECG vector of the patient or a muscle activity signal of the patient.

19. The differential voltage measuring system as claimed in claim 2, wherein
the at least one bioelectrical signal indicates a capacitive coupling between at least one of the capacitive sensor elements and the patient; and
the signal processing apparatus includes
a reference signal capture apparatus configured to capture a reference signal, and
a comparator module configured to
ascertain, on the basis of the reference signal, a corrected measurement signal, and
provide, on the basis of the corrected measurement signal, as the at least one bioelectrical signal, a signal from which a capacitive coupling between at least one of the capacitive sensor elements and the patient is derivable.

20. The differential voltage measuring system as claimed in claim 3, wherein
the at least one bioelectrical signal indicates a capacitive coupling between at least one of the capacitive sensor elements and the patient; and
the signal processing apparatus includes
a reference signal capture apparatus configured to capture a reference signal, and
a comparator module configured to
ascertain, on the basis of the reference signal, a corrected measurement signal, and
provide, on the basis of the corrected measurement signal, as the at least one bioelectrical signal, a signal from which a capacitive coupling between at least one of the capacitive sensor elements and the patient is derivable.
